(19) United States
(12) Patent Application Publication
(10) Pub. No.: US 2020/0385824 A1
(43) Pub. Date: Dec. 10, 2020

(54) DNA MARKING OF LEATHER

(71) Applicant: APDN (B.V.I.) Inc., Tortola (VG)

(72) Inventors: Michael E. Hogan, Stony Brook, NY (US); Joanna Greenwood, Oswestry (GB)

(21) Appl. No.: 16/897,862

(22) Filed: Jun. 10, 2020

Related U.S. Application Data
(60) Provisional application No. 62/859,284, filed on Jun. 10, 2019.

(51) Int. Cl. C14C 1/00; C14C 99/00; C12Q 1/6876; C14B 17/00

(52) U.S. Cl. CPC C14C 1/00; C14B 17/00; C12Q 1/6876; C14C 99/00

(57) ABSTRACT

The present invention relates generally to a system and method of tracking leather raw materials through the supply chain to create a source verifiable finished leather product through one or more applications of DNA taggants during the leather manufacturing process and the subsequent authentication of said DNA taggants.

### CROSS REFERENCE TO RELATED APPLICATIONS

The present application claims the benefit of U.S. provisional patent application No. 62/859,284 filed on Jun. 10, 2019, the contents of which is hereby incorporated by reference in its entirety.

### BACKGROUND OF THE INVENTION

### 1. Field of the Invention

The present invention relates generally to a system and method of tracking leather raw materials through the supply chain to create a source verifiable finished leather product through one or more applications of DNA taggants during the leather manufacturing process and the subsequent authentication of said DNA taggants.

### 2. Background of the Invention

The leather supply chain is globalized. Typically, the supply networks of leading brands and retailers comprise multiple tiers across numerous continents. The complexity of these supply chains causes difficulties in quality assurance, sourcing, logistics management, the tracking of provenance and counterfeiting. These issues are further exacerbated by the vast networks of independent suppliers, manufacturers and raw material sources across the leather sector.

The need for robust product traceability and transparency in the leather sector is critical. However, many global supply networks are poorly equipped to respond to such challenges. Moreover, the significant threat posed by environmental and social risks to brand value ensures that such challenges are increasing. Failure to adequately address supply chain challenges leaves companies exposed to undue risk and scrutiny which may include trade issues, prosecution and, perhaps more importantly, risk to reputation.

The leather manufacturing process encompasses a myriad of environmentally and socially sensitive aspects, including animal welfare, labor compliance and environmental pollution. The industry continues to experience increasing scrutiny in relation to deforestation, effluent discharge, use of hazardous chemicals and animal welfare. Inevitably, critical examination of the industry will grow, as will nongovernmental organizations' (NGO) influence on government policy and public opinion.

The leather industry is also subject to a bevy of national and international regulatory controls, specifically concerning upstream processes. Failure to comply with such controls can manifest in dangerous or defective products, presenting a considerable risk to every party in the supply chain, including the end-user. Moreover, the significant threat posed by environmental and social risks to brand value ensures that such challenges are increasing.

These factors serve to emphasize the importance of a thorough supply chain management system for a leather supply chain, which heretofore, has not existed. Thus, there is an unmet need for a supply chain management system configured to track and trace all aspects of a global leather supply chain, from source livestock to finished leather product.

The inventors have surprisingly discovered that DNA tags can be utilized as a taggant for authentication for all aspects of the leather supply chain, from source livestock to finished leather product, despite the harsh chemical treatments used in leather manufacturing. For example, during tanning, the animal hides may be exposed to chromium salts in a highly acidic environment. Yet despite high levels of these harsh chemicals residing in resultant leather/hides, DNA taggants can be used to indelibly tag leather and/or hides for later authentication via sequence specific DNA detection techniques.

### BRIEF SUMMARY OF THE INVENTION

In one aspect of the present invention a method of producing a source verified leather product is disclosed, said method comprising: (i) tagging a livestock with a DNA taggant, which optionally may also contain a fluorescent marker; (ii) slaughtering the livestock with the DNA taggant to produce DNA tagged rawhide; (iii) authenticating the DNA tagged rawhide; (iv) processing the authenticated rawhide to produce tanned hide, wherein during the processing of the rawhide to tanned hide, a DNA taggant is applied on or into the tanned hide; (v) authenticating the DNA taggant contained on or in the tanned hide; (vi) processing the authenticated tanned hide into a finished leather product; (vi) optionally, during processing of the tanned hide into a finished leather product, applying a DNA taggant on or into the finished leather product; and (vii) authenticating the DNA taggant on or in the finished leather product.

In another aspect, a method of marking a hide for authentication is disclosed, the method comprising: (i) adding a DNA taggant to a solvent to form a DNA taggant solution; and (ii) applying the DNA taggant solution to the hide, thereby incorporating the DNA taggant into the hide to provide a DNA-tagged hide.

In an additional aspect, a method of indelibly applying a DNA taggant to a wet blue hide is disclosed, said method comprising: (i) creating a net positive charge associated with the wet blue hide; (ii) introducing a DNA taggant solution to the net positively charged wet blue hide; and (iii) mixing the DNA taggant with the net positively charged wet blue hide to indelibly incorporate the DNA taggant on and into the wet blue hide.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating the preferred embodiments of the invention and are not to be construed as limiting the invention. In the drawings:

FIG. 1 shows the DNA amplification curve of several samples obtained from a livestock tagged with DNA taggants according to an embodiment of the invention.

FIG. 2 shows the DNA amplification curve of several samples obtained from a rawhide tagged with DNA taggants according to an embodiment of the invention.

FIG. 3 shows the DNA amplification curve of several samples obtained from a wet blue hide tagged with DNA taggants according to an embodiment of the invention.

FIG. 4 shows the DNA amplification curves of several samples obtained from a wet blue hide tagged with DNA taggants according to an embodiment of the invention after accelerated aging studies.

FIG. 5 shows the amplification curve of several samples obtained from a polyurethane finishing treatment basecoat according to an embodiment of the invention.

FIG. 6 is a flow charting detailing the method of tracking a hide from source to finished product according to an embodiment of the invention.

### DETAILED DESCRIPTION OF THE INVENTION

The following documentation provides a detailed description of exemplary embodiments of the invention. Although a detailed description as provided herein contains many specifics for the purposes of illustration, anyone of ordinary skill in the art will appreciate that many variations, equivalents and alterations to the following details are within the scope of the invention. Accordingly, the following preferred embodiments of the invention are set forth without any loss of generality to, and without imposing limitations upon, the claimed invention. Thus, the scope of the invention should be determined by the appended claims and their legal equivalents, and not merely by the preferred examples or embodiments given herein.

The word “hide” is used in this application to refer to the skin of the animal at various stages during the production of leather. For example, hide may refer to a portion of skin on a live animal, the skin that is removed from an animal after slaughtering, and the material that is processed to become leather. Additionally, a rawhide refers to a hide removed from an animal that has not undergone tanning; whereas, leather refers to a hide that has undergone the complete tanning process. Accordingly, as used herein the word “hide” encompasses livestock, rawhide, and leather.

The inventors have surprisingly discovered that DNA tags can be utilized as a taggant for authentication for all aspects of the leather supply chain, from source livestock to finished leather product, despite the harsh chemical treatments used in leather manufacturing. For example, during tanning, the animal hides may be exposed to chromium salts in a highly acidic environment. Yet despite high levels of these harsh chemicals residing in resultant leather/hides, DNA taggants can be used to indelibly tag leather and/or hides for later authentication via sequence specific DNA detection techniques.

One aspect of the invention relates to a method of marking hides used in leather production with DNA taggants for authentication. The process of authenticating hides may involve several steps of marking the material with DNA taggants verifying the origins of the material during each step in the supply chain, i.e., from initially marking and authenticating the livestock all the way to marking and authenticating the finished leather product.

For example, the livestock may be marked with a DNA taggant on the farm. Then the animals may be authenticated at the slaughtering facilities. After slaughtering, the hides may then be marked with a DNA taggant before being sent to the tannery. Upon receipt of the hides at the tannery, the hides may be authenticated using either the livestock or slaughterhouse DNA taggant. After tanning, the tanned hide may be marked again with a DNA taggant and passed onto one or more leather traders. Upon receipt, the leather traders may authenticate the hides and sell them to a finishing tannery where again the hides, containing one or more DNA taggants, may be authenticated. During processing at the finishing tannery, the hide may again be marked with a DNA taggant and sent out to be manufactured into finished products. The manufacturers, upon receipt of the DNA tagged hide, may authenticate the hide and/or manufactured finished goods to provide for full traceability throughout the supply chain.

The livestock used may be any animal with hides or skin used to make leather. For example, most leather produced comes from cattle or cow hides. Other exemplary sources of leather include, without limitation, deer, elk, pig, buffalo, goat, sheep, ox, yak, and reptiles.

### DNA Taggant

Marking of hides is accomplished by adding a DNA taggant to a solution to form a DNA taggant solution, which is then applied to the hide. Upon application, the DNA taggant is affixed to the hide to provide information about the hide such as the source of manufacture, national origin, or authenticity. Preferably, the DNA taggant is comprised of one or more non-naturally occurring DNA sequences that are adapted for use in authentication.

In one example, the DNA taggant is a double stranded DNA molecule having a length of between about 20 base pairs and about 1000 base pairs. In another example, the DNA taggant is a double-stranded DNA molecule with a length of between about 80 and 500 base pairs. In another example, the DNA taggant is a double-stranded DNA molecule having a length of between about 100 and about 250 base pairs. Alternatively, the DNA taggant can be single-stranded DNA of any suitable length, such as between about 20 bases and about 1000 bases; between about 80 bases and 500 bases; or between about 100 bases and about 250 bases. The DNA taggant can be a naturally-occurring DNA sequence, whether isolated from natural sources or synthetic; or the DNA taggant can be a non-naturally occurring sequence produced from natural or synthetic sources. All or a portion of the DNA may comprise an identifiable sequence. The preferred DNA is double-stranded DNA of a non-naturally occurring sequence. The DNA taggant may also be comprised of a mixture of amplicons produced via polymerase chase reaction (PCR) and oligonucleotides produced by oligonucleotide synthesis.

The DNA taggant may be comprised of a mixture of DNA and an ionic or non-ionic food grade surfactant. The DNA taggant may also include a food grade or non-food grade encrypted or non-encrypted optical marker. The DNA taggant may also be in non-aqueous dry powdered form or be in an aqueous solution. In an exemplary embodiment, the dry powder DNA tag is comprised of a DNA taggant and carrier complex created via a freeze drying (lyophilisation or the like) process. The carrier may be any suitable freeze dry carrier known in the art. The DNA taggant may also be alkaline activated to increase binding affinity as disclosed in U.S. Pat. No. 9,790,538 to Berrada et al. and U.S. Pat. No. 9,266,370 to Jung et al. The DNA tag may include a combination of various non-ionic emulsifiers and/or surfactants. Exemplary compounds include Span 85, sorbitan trioelate, Tween 20 and Polysorbate 20, alkyl aryl polyoxyethylene glycol, Surfadone LP-100, Surfadone LP-300 and the like; polar aprotic solvents such as Acetone, DMSO, DMPU, 2-(2-Ethoxyethoxy) ethanol and the like. The DNA taggant and/or DNA taggant solution may also include chelating agents such as ethylenediaminetetraacetic acid (EDTA).

The DNA taggant may be comprised of at least one primer binding site and one or more discrete informational units. Each discrete informational unit may be configured to impart a specific piece of data upon authentication of the DNA taggant. In an embodiment, the DNA taggant is comprised of one or more known synthetic DNA sequences, each under 500 bp in length, each of the one or more known synthetic DNA sequences including at least two primer binding sites and at least one discrete informational unit. Each discrete information unit may be between 2 bp and 100 bp in size. Authentication of the one or more discrete informational units may be accomplished via the use of one or more sequence specific DNA detection technologies. Sequence specific DNA detection technologies may include, without limitation, next generation sequencing, sanger sequencing, nanopore sequencing, quantitative PCR (qPCR), reverse transcription PCR (RT-PCR), hybridization probes, nucleic acid probes such as Taqman probes, molecular beacons, peptide, nucleic acid (PNA)-based probes, digital PCR, micro-arrays and/or any other sequence specific detection technology. A microarray may use one or more of the foregoing sequence specific DNA detection technologies on the same array. The DNA taggant may also be a combinatorial tag, comprised of more than one known synthetic DNA sequence. The combinatorial tag may be comprised of one or more amplicons and one or more oligonucleotides.

Preferably, the DNA taggant is identifiable by any suitable nucleic acid amplification and/or DNA sequence detection technique. Nucleic acid amplification may be accomplished via any technique known in the art, such as, for example, polymerase chain reaction (PCR), loop mediated isothermal amplification, rolling circle amplification, nucleic acid sequence base amplification, ligase chain reaction, or recombinase polymerase amplification. In addition, any known sequence detection and/or identification technique may be used to detect the presence of the nucleic acid taggant such as, for example, hybridization with a taggant-sequence specific nucleic acid probe, an in situ hybridization method (including fluorescence in situ hybridization: FISH), TaqMan™ probes, as well as amplification and detection via PCR, such as quantitative (qPCR)/real time PCR (RT-PCR) or TaqMan™ real-time PCR assays. Isothermal amplification and taggant sequence detection may also be performed with the aid of an in-field detection device such as the T-16 Isothermal Device manufactured by TwistDX, Limited (Hertfordshire, United Kingdom). A portable qPCR device such as the MyGo real-time PCR instrument manufactured by IT-IS Life Science Ltd (Cork, Ireland) or the like may also be utilized.

The preferred solvent to form a DNA taggant solution for application to hides at various stages of process is water or other suitable liquid chemical used in the leather manufacturing process.

Suitable exemplary ranges of DNA taggant loading for DNA tagging solution include for instance:

A range from about 0.1 nanogram (10⁻¹⁰ g) to about 15 microgram (15×10⁻⁶ g) of DNA taggant added per kilogram (10³ g) of DNA tagging solution.

A range from about 0.1 nanogram (10×10⁻¹⁰ g) to about 1 microgram (10⁻⁶ g) of DNA taggant added per kilogram (10³ g) of DNA tagging solution.

A range from about 0.1 nanogram (10×10⁻¹⁰ g) to about 100 nanograms (100×10⁻⁹ g) DNA taggant added per kilogram (10³ g) of DNA tagging solution.

A range from about 0.1 nanogram (10×10⁻¹⁰ g) to about 10 nanograms (10×10⁻⁹ g) of DNA taggant added per kilogram (10³ g) of DNA tagging solution.

A range from about 1 picograms (1×10⁻¹² g) to about 100 microgram (100×10⁻⁶ g) of DNA taggant added per kilogram (10³ g) of DNA tagging solution.

A range from about 1 femtogram (10⁻¹⁵ g) to about 1 microgram (10⁻⁶ g) of DNA taggant added per kilogram (10³ g) of DNA tagging solution.

A range from about 10 femtograms (10×10⁻¹⁵ g) to about 100 nanograms (100×10⁻⁹ g) of DNA taggant added per kilogram (10³ g) of DNA tagging solution.

A range from about 100 femtograms (100×10⁻¹⁵ g) to about 10 nanograms (10×10⁻⁹ g) of DNA taggant added per kilogram (10³ g) of DNA tagging solution.

A range from about 1 picograms (1×10⁻¹² g) to about 1 nanogram (1×10⁻⁹ g) of DNA taggant added per kilogram (10³ g) of DNA tagging solution.

A range from about 0.1 to 10 μg of DNA taggant per liter of tagging solution;

A range from about 0.12 to 15 μg of DNA taggant per kg of wet blue hide;

A range from about 36 to 360 ng of DNA taggant per kg of finished leather product; and

A range of about 4 ng to 12 ng of DNA taggant per kg of rawhide.

The amount of DNA taggant per ton of leather or other hide may range from 3 micrograms to 8 micrograms. In an embodiment, 5 micrograms of DNA taggant per ton of leather is used (5 picograms of DNA taggant per gram of leather).

In an embodiment, the DNA taggants are added to liquid processing and/or finishing treatments used in the manufacture of leather. In preferred embodiments, the DNA taggants are added to liquid processing or finish treatments in the range of 0.15% to 0.20% of the process or finishing solution total weight. In an alternative embodiment, the DNA taggants may be added to a polyurethane finishing treatment in the range of 0.10% to 0.25% or 0.15% to 0.20% of the polyurethane finishing treatment weight. The polyurethane finishing treatment containing the DNA taggants may be spray onto the hide and may be applied in a single layer or multiple layers. Polyurethane finishing treatments with DNA taggants may be applied via any sprayer known in the art, including a hand sprayer or large scale sprayer found in large leather manufacturing operations.

In an embodiment the polyurethane, finishing treatment is comprised pigments, water, wax filler, acrylate resin, an acrylate/polyurethane blend polymer and cross-linkers. The DNA taggant may be added to such a formulation as a water-based solution in the percentage of 0.10% to 0.25% or 0.15% to 0.20% of total formulation weight.

The DNA tagging solution may also include one or more additives such as chelating agents and/or fluorescent markers. Examples of chelating agents include ethylenediaminetetraacetic acid (EDTA), EGTA, BAPTA, DOTA and DTPA. Fluorescent markers may be added to assist in detecting the DNA taggant, especially in the marking of livestock and rawhide. Exemplary fluorescent markers are described in more detail in U.S. Pat. No. 10,047,282, which is hereby incorporated by reference. Examples of fluorescent markers include fluorophores. Other exemplary fluorescent markers include, but are not limited to, rhodamines, rhodols, fluoresceins, and derivatives of coumarin, cyanine, oxazine, (dipivaloylmethanato)europium(III) and lanthanides. The fluorescent marker is preferably included in an amount of less than 2% of the total DNA taggant solution.

The DNA taggant solution is then applied to the livestock, rawhide, or leather to form DNA-tagged livestock, rawhide, or leather. The DNA taggant solution may be applied to hide via any known application methods, including without limitation, spraying, painting, incorporation in an existing manufacturing process, printing, inkjet printing, laser marking rolling, coating, or curtain coating. The DNA tagging solution may also be added to another hide treatment prior to application to the hide or to the chemicals utilized in leather processing during a point in leather processing when the hide has a slight positive charge, including, by way of example, when the hide is in a slightly acid solution. In addition, leather maybe coated with finishing treatment, such as a polyurethane coat. The DNA tagging solution may be added to the polyurethane coating, or to any other finishing treatment used in the art. The DNA tagging solution may be applied and/or added to a base coat and/or topcoat of a finishing treatment.

Authentication of the Hides in the Leather Supply Chain

Once a hide is marked with a DNA taggant it can be authenticated at various points in the supply chain. Authentication involves first DNA marking the livestock, rawhide, or leather with a DNA taggant as described above. Then the livestock, rawhide, or leather is introduced into either the leather supply chain or the stream of commerce. The final step involves detecting the presence of the DNA taggant in the DNA-tagged livestock, rawhide, or leather. This step involves sampling the taggant from the marked hide, analyzing the DNA taggant, and then verifying the authenticity of the DNA taggant via a DNA sequence specific detection method.

If a fluorophore was used in the DNA tagging solution, the DNA tagged area will fluoresce under ultraviolet (UV) or other light. Otherwise the DNA tagged hide item may be randomly sampled. Obtaining a sample of the DNA taggant involves adding an optional solvent to a cotton swab and wiping the hide to obtain the sample. Preferred solvents include water, ethanol, isopropanol, and methyl ethyl ketone. The sample is then analyzed by any known sequence specific DNA detection method know in the art, optionally without the need for DNA isolation, i.e., without the conventional steps of extraction and purification. Typically, before DNA detection techniques can be employed, the DNA in the sample must be isolated and purified to allow for accurate results. Often, the steps of DNA isolation and purification are time consuming and/or costly and add complexity to the authentication process. The exclusion of these steps greatly simplifies the process of authentication and also reduces the time necessary to authenticate a hide. In the case of PCR, isolation and purification steps are usually required before the DNA can be amplified. The present methods optionally do not require these extra steps of preparing the DNA by isolation, extraction, and/or purification, though extraction may be used to increase DNA detection, if necessary. Swabbing of a hide with a cotton swab may result in destructive or non-destructive sampling of the hide.

In an exemplary embodiment in situ PCR is utilized to quickly analyze the DNA taggant obtained from a hide by the placement of part of the cotton swab containing the DNA taggant directly into the PCR reaction vessel, without prior extraction or purification steps. Utilizing in the situ PCR process, part of the cotton swab, containing a quantity of DNA taggant, is placed into the PCR reaction vessel. Extraction buffer may be added to the vessel and the sample is heated at 95° C. for 10 minutes. Thereafter, DNA amplification and/or taggant sequence detection techniques may be employed to amplify and identify the DNA taggant obtained from the hide.

For example, in a PCR-based identification method, the DNA taggant recovered from the hide is amplified by polymerase chain reaction (PCR) and resolved by gel electrophoresis, capillary electrophoresis, real-time PCR, quantitative PCR (qPCR) or the like. Since the nucleic acid sequence of the DNA taggants of the present invention are unique and specific to the tagged hide, the DNA taggant will be amplified during PCR only by use of primers having specific sequences complementary to a portion of the unique taggant DNA sequence. Through this procedure, if the examined hide carries the DNA taggant, the PCR procedure will amplify the extracted DNA to produce known and detectable amplicons of a predetermined size and a sequence. In contrast, if the sample recovered from the examined hide does not include the unique nucleic acid sequence corresponding to the taggant of the authentic item, there will likely be no detectable amplified nucleic acid product, or if the primers do amplify the recovered nucleic acid to produce one or more random amplicons, these one or more amplicons cannot have the unique taggant nucleic acid sequence and/or length from the authentic item. Furthermore, the random amplicons derived from counterfeit hides are also of random lengths and the likelihood of producing amplicons of the exact lengths specified by the taggant-specific primers is very small. Therefore, by comparing the length, sequence and/or quantity of PCR amplicons, the authenticity of DNA tagged hides can be verified, non-authentic hides can be screened and rejected, and anti-counterfeit screening purposes are then achieved. The DNA taggants may also be amplified by any other known amplification techniques such as loop mediated isothermal amplification, rolling circle amplification, nucleic acid sequence base amplification, ligase chain reaction or recombinase polymerase amplification (RPA).

The quantity of amplicons and the lengths of the amplicons can be determined after any molecular weight or physical dimension-based separation, such as for instance and without limitation, gel electrophoresis in any suitable matrix medium for example in agarose gels, polyacrylamide gels or mixed agarose-polyacrylamide gels, or the electrophoretic separation can be in a slab gel or by capillary electrophoresis. Mass spectrometry-based techniques can also be utilized.

In addition, any known sequence detection and/or identification technique may be used to detect the presence of the DNA taggant such as, for example, hybridization with a marker-sequence specific nucleic acid probe, an in situ hybridization method (including fluorescence in situ hybridization: FISH), TaqMan probes, as well as amplification and detection via PCR, such as quantitative (qPCR)/real time PCR (RT-PCR). Isothermal amplification and taggant sequence detection may also be performed with the aid of an in-field detection device such as the T-16 Isothermal Device manufactured by TwistDX, Limited (Hertfordshire, United Kingdom). Interrogation may also be accomplished via various in-field techniques as disclosed in Jung et al. (U.S. Ser. No. 14/471,722) as well as through the use of infield microarray system utilizing a sequence specific quenched florescent probe system. Interrogation may also be accomplished via the use of portable qPCR devices such as the MyGo real-time PCR instrument manufactured by IT-IS Life Science Ltd (Cork, Ireland).

### Source Verified Leather Supply Chain

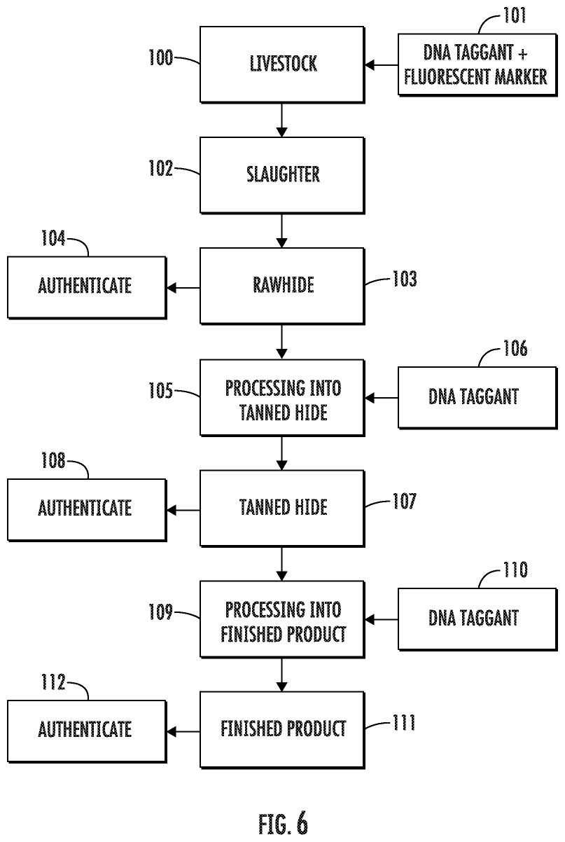

In an embodiment, a method of producing a source verified leather product is disclosed. The method utilized DNA taggants, applied at different point in the leather product manufacturing supply chain to provide for a finished product that can be source verified. As shown in FIG. 6, the method comprises the following steps: (i) tagging a livestock (100) with a DNA taggant, which optionally may also contain a fluorescent marker (101); (ii) slaughtering the livestock (102) with the DNA taggant to produce DNA tagged rawhide (103); (iii) authenticating the DNA tagged rawhide (104); (iv) processing the authenticated rawhide (105) to produce tanned hide (107), wherein during the processing of the rawhide to tanned hide (105), a DNA taggant is applied on or into the tanned hide (106); (v) authenticating the DNA taggant contained on or in the tanned hide (108); (vi) processing the authenticated tanned hide (109) into a finished leather product (111); (vi) optionally, during processing of the tanned hide into a finished leather product (109), applying a DNA taggant on or into the finished leather product (110); and (vii) authenticating the DNA taggant (112) on or in the finished leather product (111).

DNA taggant application during the processing of the rawhide into tanned hide (105) may be performed at the wet blue stage of processing. Application of the DNA taggant may be performed on a wet blue hide carrying a positive charge imparted by a slightly acid pH. After application to the wet blue hide, the DNA taggant may be resident on the surface and/or within the internal structure of the hide. Authentication (104, 108 and 112) of the hides may be performed by any DNA sequence specific detection method known in the art. In a preferred embodiment authentication is performed via a portable qPCR device. During the processing of a tanned hide into a finished product (109), the DNA taggant (110) may be applied to the hide as part of a processing or finishing treatment. In a preferred embodiment, the DNA taggant is applied the hide as part of a polyurethane finishing treatment. The DNA taggant may be between 0.10% to 0.25% or 0.15% to 0.20% of the polyurethane finishing treatment total formulation weight. The DNA taggant (110) added during the processing a tanned hide into a finished product (109) may be the same DNA taggant as added earlier in the leather manufacturing process (101 and 106) or it may be a different DNA taggant not previously added during leather processing.

Examples have been set forth below for the purpose of illustration and to describe the best mode of the invention at the present time. The scope of the invention is not to be in any way limited by the examples set forth herein.

### EXAMPLES

### Example 1—Marking of Livestock with DNA Taggants

Aerosol canisters containing a DNA taggant solution with U.V. fluorescent optical marker spray was used for marking livestock. The spray were applied to live cows for subsequent recovery and authentication from the hide after slaughter.

A sufficient spray was applied to each side of the rump of each cow, towards the spine. Prior to marking the area to be sprayed was cleaned of any loose surface dirt. The marked hide area was colorless under normal lighting conditions, however the marked areas were clearly visible under UV light immediately post-application. The tagged cows where then released back into the environment with no special follow-on treatment prior to slaughter.

After slaughter, the hide was spread out and the DNA taggant marked areas identified under UV light. The previously marked areas where readily visible. Cotton swabs were taken from each of the fluorescent areas identified on the hide. Prior to sampling the surface of the hide was briefly patted dry. No further washing or cleaning of the hide was performed. Samples were taken using cotton swabs moistened with acetone or deionised water. Care was taken to ensure that swabs contained transferred material. This was accomplished by monitoring fluorophore transfer onto the swab tips.

The top <1 mm of the cotton swabs were cut using a sterile razor blade and placed into a reaction vessel containing extraction solution. The tubes were then heated using the SigNify® IF portable qPCR device (Applied DNA Sciences, Inc., Stony Brook, N.Y.) to extract the DNA from the swabs. An aliquot of the resulting extraction product was placed into tubes containing a proprietary qPCR (PCR) master mix to form an extraction sample. Further samples were created by dilution of the extraction product. All samples were run on the SigNify® IF portable qPCR DNA reader. Each analytical run contained up to 16 samples (including positive and negative controls) and was completed in less than one hour.

All swabs taken from the fluorescent areas of the hide were successfully authenticated, with DNA detected in all samples via qPCR analysis. During qPCR analysis, the DNA taggants removed from the hide are amplified during a series of heating cycles. Each cycle leads to an approximate doubling in the quantity of the targeted DNA clone. As the DNA is copied (or amplified), each molecule is labelled with a fluorescent tag, allowing the amount of DNA present to be monitored. The amount of fluorescence present (or intensity) is recorded using relative fluorescent units (RFUs). Once the level of fluorescence reaches the threshold for detection, the signal is seen to increase exponentially with each cycle. The amount of DNA present in the original sample is represented by a Cq (Ct) value. Cq values represent the inflexion point in PCR amplification where the amount of DNA reaches the threshold for detection. The Cq value is inversely proportional to the amount of DNA present in the original sample, thus a higher Cq value represents a lower level of DNA recovery.

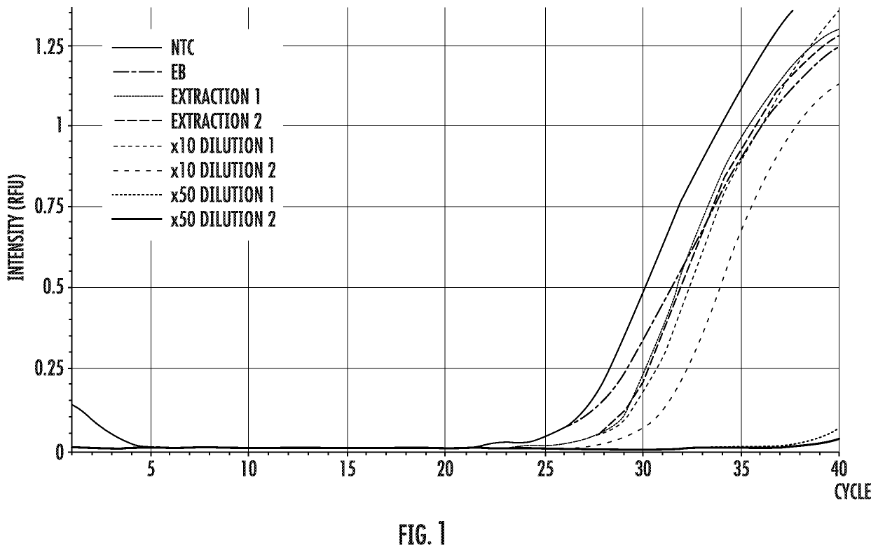

As shown in FIG. 1, the Cq values found for the cotton swabs from the hides showed that significant DNA was present in all samples, allowing for reliable post slaughter detection of DNA tags applied to hides pre-slaughter.

### Example 2—Marking of Raw Hide at Slaughterhouse with DNA Taggants

Raw hides were marked with a DNA taggant solution containing a U.V. fluorescent optical marker, and the hide was preserved by salting. The results of this testing are applicable to both DNA taggants applied to livestock and to raw hide marked at the slaughterhouse. A fresh hide was obtained and marked with a DNA taggant and solution. Cotton swabs were taken from the DNA marked areas for DNA authentication up to 6 months after marking. To minimize the amount of salt collected by the swabs, excess salt was brushed away from the surface of the hide, and the marked area was rinsed and dried prior to sampling. The removal of excess salt is required to increase the efficiency of amplification during the PCR process; the salt itself does not adversely impact the DNA taggants.

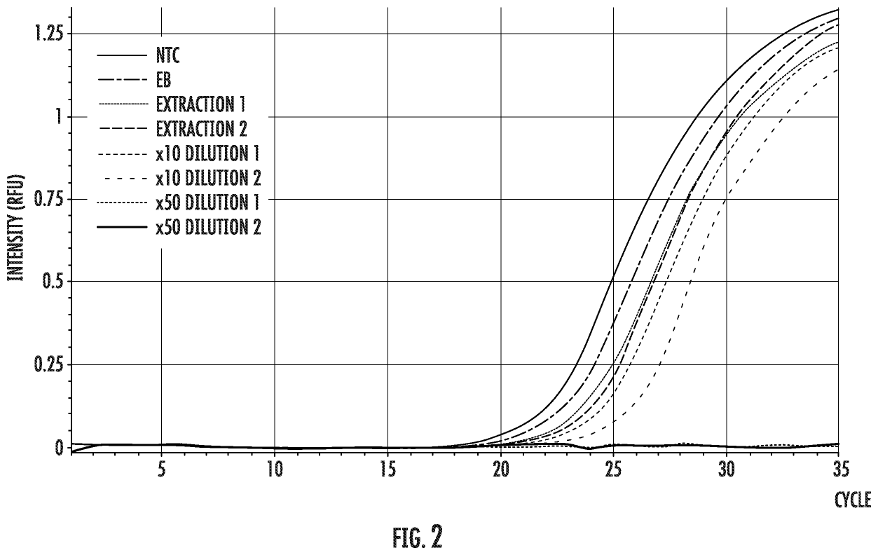

After 6 months, the hide was processed through to the wet blue stage to confirm removal of the visual marking aspects of the DNA tagging solution, along with the hair during the de-hairing process. Further details of the wet blue process used can be found in Example 3. The DNA tagging solution was still clearly visible under UV light after six months' storage and sampling. Swabs were taken for authentication from the visible areas using the SigNify® IF portable DNA reader during the six month's storage as salted hide. The swabs taken from the hide were analyzed after performing an extraction of the DNA from the cotton swab placed in a PCR reaction vessel and after dilution of the extracted product. This methodology allows the impact of potential PCR inhibitors, such as salt, to be reduced. Further details of the DNA testing methods used and data interpretation can be found above in Example 1._As shown in FIG. 2, the DNA taggants were successfully authenticated in all samples taken from the hides.

### Example 3—Marking of Wet Blue Hide to Finished Hide with DNA Taggants

DNA marking of the wet blue (or wet white) provides traceability throughout this critical stage in the manufacture of the leather. The term wet blue refers to moist chrome-tanned leather, DNA marking can equally be applied to leather tanned using other methods, such as aldehyde/syntan tanning (wet white), aluminum, or zirconium tanning, vegetable tanning, or oil tanning. In this phase, the leather is tanned, but neither dried, dyed nor finished. DNA marking with DNA taggants of the wet blue was carried out under real world conditions at both a small-scale research tannery and during commercial-scale tanning.

After salting, the hides were stored before soaking to remove surface dirt. The hide was then fleshed prior to liming. The limed hide was weighed to give a base weight to be used in calculating the quantities of chemicals required for the remaining tanning process. The limed weight was also used to determine the amount of DNA taggant to be added to the final rinse. The DNA taggant was used at the concentration of between 0.1 to 10 μg per kg of limed hide. The limed hide was delimed, bated and rinsed. The pH was lowered prior to tanning using a pickle. Tanning was carried out using chromium salts. Once the chromium had fully penetrated the hide, the pH was raised by basification to around 4 in both the float and the hide.

After draining the tanning mixture, the drum was re-filled using clean water containing fungicide and a cleating agent. This was dissolved using the mechanical action of the drum. A slightly acidic pH persisted, imparting a cationic (positive) charge to the hide, which is predominantly comprised of collagen. A pH of between 3.5 and 6.9 is preferred. The DNA taggants, due to its phosphate groups has a net negative charge under the conditions of the instant methodology. This fact causes the DNA taggants (with their net negative charge) to bind to and penetrate the hide (which at this point in the tanning process has a net positive charge).

Two unique DNA taggant of known sequences were added to the rinse. The DNA was provided in a water-based solution which was added to the drum at a volume ratio of concentrate to rinse of 0.05% (DNA1) and 0.005% (DNA2). The use of different levels of DNA taggants enabled an assessment of the optimum amount of DNA taggant to be used for commercial applications. After DNA taggant inoculation of the rinse, the wet blue was drummed for 30 minutes to 1 hour before the excess liquid was drained and the wet blue sampled for the adherence of the DNA taggants. The net positive charge and porous nature of the hide during this stage of processing caused the negatively charged DNA taggant to penetrate into all aspects of the hide, thereby indelibly marking the hide with DNA.

To prove that the DNA taggant has homogenously and indelibly marked all aspects of the hide, a grid system was used for sampling. This sampling strategy was designed to ensure that all areas of the wet blue hide had been uniformly marked with DNA taggants and ensure that the sections of hide used for further processing into leather and accelerated ageing studies were representative of the whole. Samples were taken by swabbing using a dry cotton swab (sufficient moisture was present on the hide to dampen the swab without the use of water or other solvents). Samples from the flesh side and splits were taken from the same location on the reverse. Homogeneity was established by sampling all locations.

To confirm the presence of the DNA taggants, the swab tips were cut and placed into an extraction solution prior to heating to extract DNA into the liquid phase from the cotton swab. A sample of the liquid was then added to the PCR mix for amplification as an extraction sample. All samples were authenticated using the portable SigNify® IF portable DNA reader. Details of the DNA testing methods used and data interpretation can be found above in Example 1.

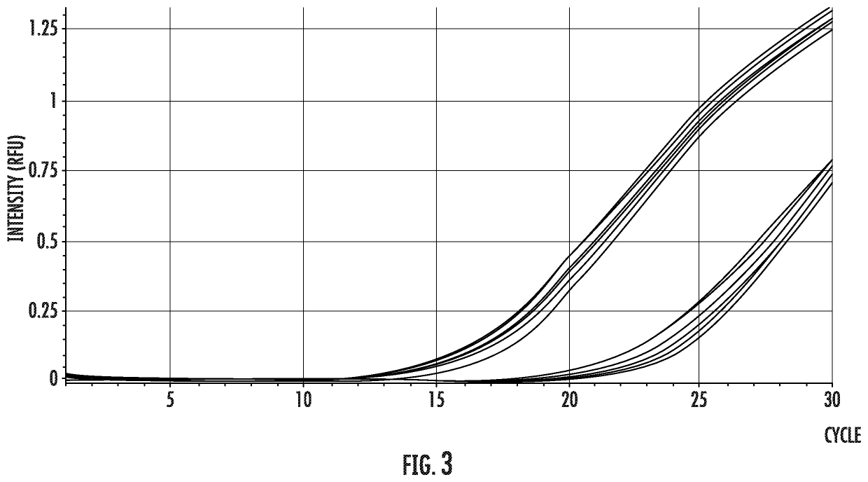

As shown in FIG. 3, the DNA taggants were successfully authenticated in all samples from the marked wet blue over a six-month stability testing period, with the DNA taggant remaining stable throughout the wet blue hide during the entire test period. Homogeneity of DNA taggant marking was found to be high across the hide, with no significant variation between samples taken from different locations on the grain and flesh sides of the hide.

The wet blue was then sammed to remove excess moisture prior to splitting. Samples were taken from the original outer surfaces (grain side and flesh side) and compared to swabs from the inner sides (grain split and drop split) to assess the degree of penetration of DNA taggants into the wet blue hide. The DNA taggants were successfully recovered from all samples of split wet blue.

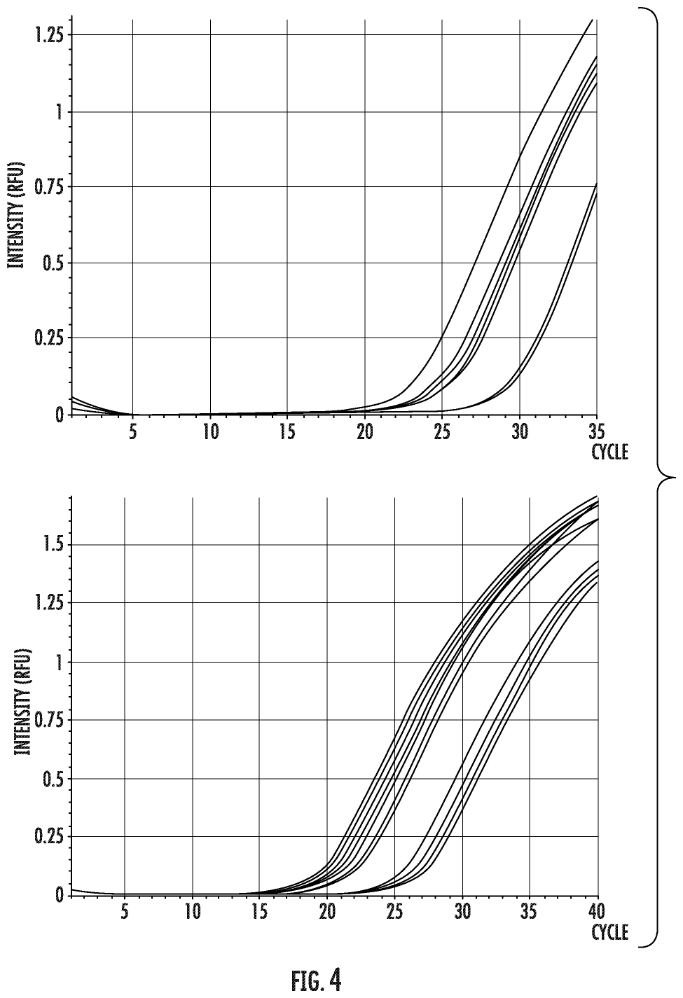

In addition, accelerated ageing testing was carried out to provide further information regarding the performance of the DNA taggant in the wet blue under specific environmental conditions. Swabs moistened with water were used to sample DNA from the dried wet blue. As shown in FIG. 4, the DNA taggants were successfully authenticated after UV and heat ageing tests.

Photomicrographs taken of the wet blue show the relatively open structure of the leather which, along with the hide's net positive charge during the DNA taggant application, allowed penetration of the DNA taggant into the center of the full substance hide. According to the literature, the porosity of leather can range from 7 Å to 150 μm, with chromium tanned leather porosity being towards the higher end of the range. This pore size and positive charge allowed the DNA taggants to diffuse into the surface pores of the wet blue hide as the DNA taggants used were less than tens of nanometers in size.

### Example 4—Marking with DNA Taggants During Retanning

Split wet blue was re-tanned and processed to finished and coated leather (grain split) and suede (drop split) at a commercial leather processing facility. Samples were taken from the hide after each stage of processing, to enable the persistence of the DNA taggant to be monitored. These included samples after splitting; wetback; neutralization; rinsing; retanning; dyeing, fixing and rinsing.

For drop split processing, the drop split was DNA-tagged with an additional DNA taggant during the second rinse. This provides a method of marking leathers which may not have any additional finish applied. Water was added to the drum, with a chelating agent as required. This was drummed at 20° C. for 5 minutes. The DNA was then added to the drum and drummed for a further 30 minutes, also at 20° C., before draining. Two DNA taggants were added to the rinse. The DNA taggant was provided in a water-based DNA taggant solution, which was added to the drum at a volume ratio of concentrate to rinse of 0.05% (DNA1) and 0.005% (DNA2). After marking, the leather was dried to a moisture content of between 6% and 8%.

For grain split processing, A new DNA taggant was added to a polyurethane base coat. The DNA taggant was added to water, with chelating agent as required and shaken to mix to form a DNA tagging solution. This DNA tagging solution was then added to the base coat formulation and shaken to mix prior to filtering. The coating with the DNA taggants was applied by spraying prior to drying and curing. Polyurethane-based intermediate and top coats, with no DNA taggants added, were also applied to the leather.

Samples were taken from the hide during retanning. Samples were taken from the drop split (suede) using water-moistened swabs. The polyurethane finish was sampled using swabs moistened with acetone. Samples were run following an extraction protocol as described in Example 1. All samples were authenticated using the portable SigNify® IF portable DNA reader. DNA taggants were successfully authenticated from both drop slip and grain split processing samples.

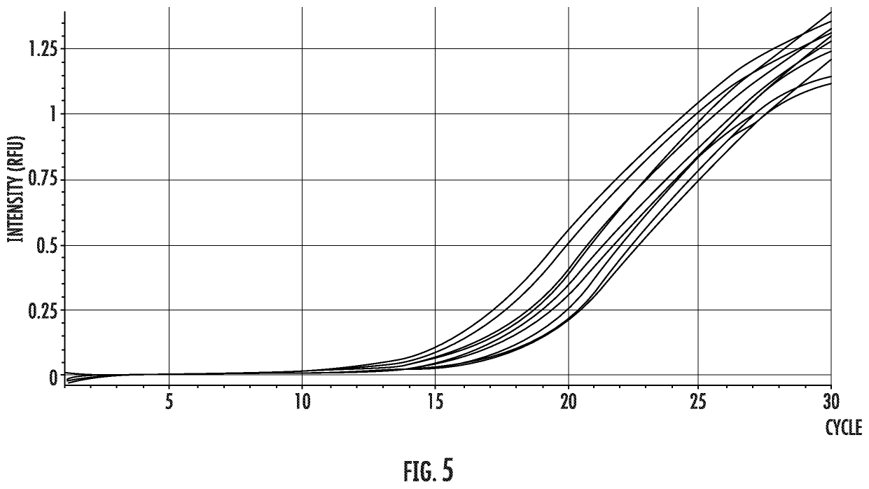

In addition, as seen in FIG. 5, the DNA taggants added to the polyurethane base coat was authenticable. There was no significant change in the amount of DNA recovery from the finished leather over the 12-week sampling period.

### Example 5—Authentication of DNA Taggants in Finished Goods

A further study was conducted, subjecting a DNA tagged hid to representative finished goods manufacturing conditions. The following tests were carried out to assess the effect of heat, steam and freezing temperatures on the recovery and authentication of the DNA taggants used in finished leather: (i) leather placed in a freezer at −8° C. for 15 minutes; (ii) Exposed to steam from boiling water for 5 minutes; (iii) storage in a laboratory oven at 130° C. for 10 minutes; (iv) storage in a laboratory oven at 120° C. for 75 minutes; (v) exposure to a hot plate at 150° C. for 30 second (pressed down firmly); (vi) exposure to a hot plate at 100° C. for 60 seconds (pressed down firmly). After testing, swabs were taken from the drop split side of the suede leather using cotton swabs moistened with water. Samples were also taken from the coated leather using cotton swabs moistened with acetone Swabs were prepared for authentication as described above in Example 1.

For the suede samples, the DNA taggants were positively authenticated on all samples after the manufacturing simulation tests. The results obtained provide reassurance that the conditions likely to be experienced during finished goods manufacture will not degrade authentication of DNA taggant added to suede via a final rinse.

For the leather samples, the DNA taggants were positively authenticated from polyurethane-coated leather samples after exposure to extremes of temperature and steam. The results obtained provide reassurance that the conditions likely to be experienced during finished goods manufacture will not degrade authentication of DNA taggant added to leather in a polyurethane coating.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention.

Although the invention has been described with reference to the above examples and embodiments, it is not intended that such references be constructed as limitations upon the scope of this invention except as set forth in the following claims.

What is claimed is:

1. A method of marking a hide for authentication, comprising:
adding a DNA taggant to a solvent to form a DNA taggant solution; and
applying the DNA taggant solution to the hide, thereby incorporating the DNA taggant into the hide to provide a DNA-tagged hide.

2. The method according to claim 1, wherein the hide is a live animal, a rawhide, or leather.

3. The method according to claim 1, wherein the solvent is chosen from the group consisting of water, acetone, oil or a polymer.

4. The method according to claim 1, wherein a chelating agent is added to the solvent.

5. The method according to claim 4, wherein the chelating agent is ethylenediaminetetraacetic acid (EDTA).

6. The method according to claim 1, wherein a fluorescent marker is added to the solvent.

7. A method of tracking a hide through a leather supply chain, comprising:
tagging a livestock with a DNA taggant;
slaughtering the livestock with the DNA tag to produce DNA tagged rawhide;
authenticating the DNA tagged rawhide;
processing the authenticated rawhide to produce tanned hide, wherein during the processing of the rawhide to tanned hide, a DNA taggant is applied on or into the tanned hide;
authentication the DNA taggant contained on or in the tanned hide;
processing the authenticated tanned hide into a finished leather product;
optionally, during processing of the authenticated tanned hide into a finished leather product, applying a DNA taggant on or into the finished leather product; and
authenticating the DNA taggant on or in the finished leather product.

8. The method of claim 7, wherein authentication of the DNA taggant is performed via a portable qPCR device.

9. The method of claim 7, wherein the DNA taggant is applied on or into the tanned hide via DNA taggant application to a net positively charged wet blue hide.

10. The method of claim 7, wherein the DNA taggant applied to the livestock includes a fluorescent marker.

11. The method of claim 8, wherein in situ PCR is utilized in conjunction with the portable qPCR device.

12. A method of indelibly applying a DNA taggant to a wet blue hide, comprising:
creating a net positive charge associated with the wet blue hide;
introducing a DNA taggant solution to the net positively charged wet blue hide; and
mixing the DNA taggant with the net positively charged wet blue hide.

13. The method of claim 12, wherein a net positive charge is imparted to the wet blue hide by an acidic pH.

14. The method of claim 13, wherein the acidic pH is between 3.5 and 6.9.

15. The method of claim 12, wherein the DNA taggant solution and wet blue hide are mixed in a drum.

16. The method of claim 12, wherein the amount of DNA taggant mixed with the net positively charged wet blue hide is between 0.1 to 10 μg per kg of hide.

17. The method of claim 12, wherein the DNA taggant is introduced to the net positively charged wet blue hide in a water solution at an amount of between 0.1 μg to 10 μg per kg of hide.
